(19) United States
(12) Patent Application Publication
(10) Pub. No.: US 2015/0140085 A1
(43) Pub. Date: May 21, 2015

(54) FORMULATIONS COMPRISING IBRUTINIB

(71) Applicant: PRINCIPIA BIOPHARMA INC., South San Francisco, CA (US)

(72) Inventor: David Michael Goldstein, Redwood City, CA (US)

(73) Assignee: Principia Biopharma Inc., South San Francisco, CA (US)

(21) Appl. No.: 14/403,927

(22) PCT Filed: Jun. 26, 2013
(86) PCT No.: PCT/US2013/047958

§ 371 Date: Nov. 25, 2014

Related U.S. Application Data
(60) Provisional application No. 61/666,562, filed on Jun. 29, 2012.

(51) Int. Cl. A61K 9/28; A61K 47/32; A61K 9/50; A61K 45/06; A61K 31/519; A61K 9/48

(52) U.S. Cl. CPC A61K 9/2846; A61K 31/519; A61K 9/4825; A61K 9/4808; A61K 9/5026; A61K 9/4891; A61K 45/06; A61K 47/32
USPC 424/452; 514/262.1; 424/465; 424/497; 424/142.1; 424/133.1

(57) ABSTRACT

Oral pharmaceutical formulations of ibrutinib and/or a pharmaceutically acceptable salt thereof, methods for their administration, process of their production, and use of these formulations for the treatment of diseases treatable by ibrutinib such as cancer, inflammatory diseases, and autoimmune diseases.

The present disclosure provides certain oral pharmaceutical formulations of ibrutinib, certain methods for their administration, certain processes of their production, and certain uses of these formulations for the treatment of diseases treatable by ibrutinib such as cancer, inflammatory diseases, and autoimmune diseases.

Bruton's tyrosine kinase (BTK) is a member of the Tec tyrosine kinase family. BTK is expressed in most hematopoietic cells such as B cells, mast cells, and macrophages, but not in T cells, natural killer cells, and plasma cells. BTK plays a role in the development and activation of B cells. Mutations in the human BTK gene cause the inherited disease X-linked agammaglobulinemia (XLA), with lack of peripheral B cells and low levels of serum Ig. In XLA, the primary immune deficit is B cell specific. The development of drugs which inhibit BTK can have therapeutic significance in the treatment of both B cell-related hematological cancers (e.g. non-Hodgkin lymphoma (NHL) and B cell chronic lymphocytic leukemia (B-CLL), and autoimmune diseases (e.g. rheumatoid arthritis, Sjogrens syndrome, IBD, lupus, and asthma).

PCI-32765 (ibrutinib) is disclosed in U.S. Pat. No. 7,514,444, issued on Apr. 7, 2009, and has the following structure:

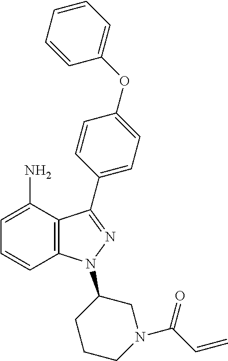 [SMILES: C=CC(=O)N1CCC[C@@H](n2nc(-c3ccc(Oc4ccccc4)cc3)c3c(N)cccc32)C1]

Ibrutinib is an orally available drug that targets Bruton's tyrosine kinase (BTK). Ibrutinib is an irreversible small molecule BTK inhibitor that is in Ph Ib/II of clinical trials in a variety of B-cell malignancies including chronic lymphocytic leukemia (CLL), small lymphocytic lymphoma (SLL), mantle cell lymphoma (MCL), diffuse large B-cell lymphoma (DLBCL) and multiple myeloma (cancer of plasma cells, a type of white blood cell present in bone marrow). At present ibrutinib is administered orally in clinical trials, via the gastrointestinal tract, at high clinical doses (420 mg/day or 840 mg/day) to patients with CLL and SLL to obtain the desired thereapeutic effect. The need for such high doses of ibrutinib may be due to low bioavailability (the oral bioavailability of ibrutinib is reported to be 22.8% in rats) and may be responsible for the adverse side effects associated with the use of ibrutinib such as nausea or emesis, dizziness and diarrhea. Moreover, low bioavailability results in more variable absorption and potential variability of the desired therapeutic response.

As stated above, at present ibrutinib is administered orally, via the gastrointestinal tract, at high clinical doses (420 mg/day or 840 mg/day) to patients to obtain the desired clinical benefit. It is presently disclosed that when ibrutinib is administered intraduodenally versus via the gastrointestinal tract in rats, the oral bioavailability of ibrutinib unexpectedly increased from 21% to 100% as determined by AUC. This unexpected increase in oral bioavailability of ibrutinib can translate into a number of desirable practical benefits. The increase in oral bioavailability should enable administration of ibrutinib at a significantly lower therapeutically effective dose than is currently being used. The lower variability associated with this greater bioavailability should lead to a more reliable therapeutic response as well as more predictable drug absorption. And avoidance of exposure of Ibtrutinib to the stomach and/or use of lower therapeutically effective dose of ibrutinib can reduce or altogether eliminate potential adverse side effects of this drug such as diahrrea, nausea or emesis, and dizziness. U.S. Pat. No. 7,514,444, mentioned above, discloses administration of 0.02-5000 mg/kg and 1-1500 mg of ibrutinib/per day and in clinical trials 420 or 840 mg/day of ibrutinib is being administered to the patients with CLL and SLL. There is no reasonable expectation in the art that ibrutinib can be adminstered orally at lower efficacious doses to the patients with CLL and SLL, particularly as evidenced by the 420 or 840 mg/day of ibrutinib being administered in clinical trials to those patients. Moreover, other than for active agents that are unstable in the stomach or at acidic pH delivery of any active agent with low bioavailability further along in the gastrointestinal tract reduces the path length for drug absorption and would be expected to reduce bioavailability. Therefore, it was unexpected to achieve delivery of ibruntinib directly to the small intestine with greater bioavailability.

Accordingly, in one aspect, the present disclosure provides a solid oral dosage form comprising:

(i) ibrutinib and/or a pharmaceutically acceptable salt thereof;

(ii) means for release of ibrutinib in the intestine; and

(iii) at least one pharmaceutically acceptable excipient.

In one embodiment of above aspect, ibrutinib and/or a pharmaceutically acceptable salt thereof is released in the small intestine. In another embodiment, ibrutinib and/or a pharmaceutically acceptable salt thereof is released to a region of the intestine in which the pH is about 5, or 5, or greater than 5. In another embodiment, said ibrutinib and/or a pharmaceutically acceptable salt thereof is released to a region of the intestine in which the pH is about 5.5, or greater than about pH 5.5. For example, the release is in one or more of the duodenum, jejunum, ileum, and colon. In one embodiment, the release is in one or more of the duodenum, jejunum, or ileum. In one embodiment, the release to the above regions of the intestine is achieved by coating ibrutinib and/or a pharmaceutically acceptable salt thereof or the dosage form containing ibrutinib and/or a pharmaceutically acceptable salt thereof with at least one coating chosen from enteric coatings and non-enteric time-delayed release coatings. In one embodiment, the release to the above regions of the intestine is achieved by coating ibrutinib and/or a pharmaceutically acceptable salt thereof or the dosage form containing ibrutinib and/or a pharmaceutically acceptable salt thereof with at least one coating chosen from enteric coatings. In one embodiment, the release to the above regions of the intestine is achieved by coating ibrutinib and/or a pharmaceutically acceptable salt thereof or the dosage form containing ibrutinib and/or a pharmaceutically acceptable salt thereof with at least one coating chosen from enteric coatings wherein the enteric coatings are chosen from polymeric coatings. In another embodiment, the enteric coating is is an anionic polymer such as polymethacrylates (e.g., methacrylic acid ethacrylate poly, methacrylic acid methyl methacrylate poly); cellulose-based polymers (e.g., cellulose acetate phthalate (CAP), cellulose acetate trimellitate (CAT), cellulose acetate succinate (CAS), hydroxypropylmethyl-cellulose phthalate (HPMCP), and hydroxypropylmethylcellulose acetate succinate (HPMCAS)) or polyvinyl derivatives such as polyvinyl acetate phthalate (PVAP). When a non-enteric coating is employed, the time-delayed release dosage forms are administered in fasted state and the time-delayed release coating is designed to erode, burst, or become hightly permeable in about 0.3 to about 3 hours or in about 0.5 to about 2 hours after administration to release ibrutinib and/or a pharmaceutically acceptable salt thereof.

In a second aspect, the present disclosure provides a solid oral dosage form comprising:

(i) ibrutinib and/or a pharmaceutically acceptable salt thereof;

(ii) means for increasing the oral bioavailability of ibrutinib, as measured by the area under the curve (AUC), as compared to when said ibrutinib and/or said pharmaceutically acceptable salt thereof are administered in an immediate release dosage form; and

(iii) at least one pharmaceutically acceptable excipient.

In one embodiment of the second aspect, the increase in the oral bioavailability of ibrutinib and/or a pharmaceutically acceptable salt thereof is due to the release of the ibrutinib and/or a pharmaceutically acceptable salt thereof in the intestine. In another embodiment of the second aspect, the increase in the oral bioavailability of ibrutinib and/or a pharmaceutically acceptable salt thereof is due to the release of the ibrutinib and/or a pharmaceutically acceptable salt thereof in the small intestine. In another embodiment of the second aspect, ibrutinib and/or a pharmaceutically acceptable salt thereof is released in one or more of the duodenum, jejunum, or ileum. In one embodiment, the release to the above regions of the intestine is achieved by coating ibrutinib and/or a pharmaceutically acceptable salt thereof or a a dosage form containing ibrutinib and/or a pharmaceutically acceptable salt thereof with at least one coating chosen from enteric coatings and a non-enteric time-delayed release coatings. When the delayed release dosage forms are administered in fasted state, the time-delayed release coating is designed to erode, burst, or become very permeable in about 0.3 to about 3 hours or in about 0.5 to about 2 hours after administration to release ibrutinib and/or a pharmaceutically acceptable salt thereof. When the dosage form comprised of said compound is coated with a non-enteric coating, it is generally administered in the fasted state to avoid variability or delays in gastric emptying with meals and the resulting variability in the initiation of efficacious plasma levels.

In a third aspect, the present disclosure provides a solid oral dosage form comprising:

(i) ibrutinib and/or a pharmaceutically acceptable salt thereof;

(ii) at least one coating chosen from enteric coatings and non-enteric time-delayed release coatings; and

(ii) at least one pharmaceutically acceptable excipient.

In one embodiment, the said at least one coating is chosen from enteric coatings. In one embodiment, the said at least one coating is chosen from polymeric coatings. In one embodiment, the said at least one coating is chosen from enteric coatings where the enteric coating is a polymer which erodes to release ibrutinib and/or a pharmaceutically acceptable salt thereof at about pH 5 and above. In another embodiment, ibrutinib and/or a pharmaceutically acceptable salt thereof is released at about pH 5.5 and above or from about 5.5 to about 6.5. In yet another embodiment of the third aspect, ibrutinib and/or a pharmaceutically acceptable salt thereof is released in one or more of the duodenum, jejunum, or ileum. In one embodiment of the third aspect and embodiments contained therein the dosage form is coated. In one embodiment of the third aspect and embodiments contained therein said ibrutinib and/or said pharmaceutically acceptable salt thereof are coated.

In a fourth aspect, the present disclosure provides a solid oral dosage from comprising:

(i) about 20 mg to about 450 mg of ibrutinib and/or a pharmaceutically acceptable salt thereof;

(ii) at least one coating chosen from an enteric coating and/or a non-enteric time-delayed release coating; and

(iii) at least one pharmaceutically acceptable excipient;

wherein said oral dosage form increases the oral bioavailability, as measured by the area under the curve (AUC), of said ibrutinib and/or said pharmaceutically acceptable salt thereof by at least 20% as compared to the bioavailability obtained from an immediate release solid oral dosage form comprising the same dose of said ibrutinib and/or said pharmaceutically acceptable salt thereof and said at least one pharmaceutically acceptable excipient under the same conditions. In one embodiment, the increase in bioavailability is at least 30%, 35%, 40%, 45%, 50%, 55%, 60%, 65%, 70%, 75%, 80%, 85%, 90%, 95%, or 100%. In another embodiment the increase in bioavailability is independently at least 70%, or 75%, or 80%, or 85%, or 90%, 95% or 100%.

In one embodiment of the first to fourth aspect and embodiments contained therein, the dosage form contains from about 20 mg to about 450 mg of said ibrutinib and/or said pharmaceutically acceptable salt thereof. In another embodiment of the fourth aspect and embodiments contained therein, the dosage form contains from about 20 mg to about 420 mg of said ibrutinib and/or said pharmaceutically acceptable salt thereof. In another embodiment of the fourth aspect and embodiments contained therein, the dosage form contains from about 20 or 30 mg to about 300 or 350 mg of said ibrutinib and/or said pharmaceutically acceptable salt thereof. In another embodiment of the fourth aspect and embodiments contained therein, the dosage form contains from about 50 mg to about 200, or 220, or 250 mg of said ibrutinib and/or said pharmaceutically acceptable salt thereof.

In one embodiment, the solid oral dosage forms disclosed above are coated with at least one coating chosen from enteric coatings and non-enteric time-delayed release coatings. Within this embodiment, in one embodiment, the at least one coating is chosen from enteric coatings. Within the above embodiments, the enteric coatings are chosen from polymeric coatings.

In another embodiment, the solid oral dosage form disclosed above comprise ibrutinib and/a pharmaceutically acceptable salt thereof that are coated with at least one coating chosen from enteric coatings and non-enteric time-delayed release coatings. Within this embodiment, in one embodiment, the at least one coating is chosen from enteric coatings. Within the above embodiments, the enteric coatings are chosen from polymeric coatings. Within the above embodiments, the enteric coating is an anionic polymer such as polymethacrylates (e.g., methacrylic acid ethacrylate poly, methacrylic acid methyl methacrylate poly); cellulose-based polymers (e.g., cellulose acetate phthalate (CAP), cellulose acetate trimellitate (CAT), cellulose acetate succinate (CAS), hydroxypropylmethyl-cellulose phthalate (HPMCP), and hydroxypropylmethylcellulose acetate succinate (HPMCAS)) or polyvinyl derivatives such as polyvinyl acetate phthalate (PVAP).

In one embodiment, the solid oral dosage forms are a tablet or capsule. When the dosage form is capsule, ibrutinib and/or a pharmaceutically acceptable salt thereof can be present in a non-solid form. In another embodiment, the solid oral dosage form disclosed above comprises ibrutinib.

The therapeutically effective amount of ibrutinib and/or a pharmaceutically acceptable salt thereof when administered into the intestine by bypassing the stomach can be from about 20 mg per day to about 450 mg/day, or 20 mg/day to about 420 mg/day; or about 20 mg/day or 30 mg/day to about 300 or 350 mg/day; or about 30 or 50 mg/day to about 200, or 220 or 250 mg/day; or about 30 or 50 mg/day to about 100 or 150 mg/day and can be administered in single or multiple doses. Accordingly, any of the formulations disclosed herein can contain from about 5, 10, 15, 20, 25, 30, 35, 40, 45, 50, 55, 60, 65, 70, 75, 80, 85, 90, 95, 100, 110, 115, 120, 125, 130, 135, 140, 145, 150, 155, 160, 175, 170, 175, 180, 185, 190, 195, 200, 225, 250, 300, 325, 350, 375, 400, 425, or 450 milligrams of ibrutinib or a pharmaceutically acceptable salt thereof. In one embodiment, the tablets or capsules can contain about 20, 25, 30, 50, 75, 100, 150, 200, or 220 milligrams of ibrutinib and/or a pharmaceutically acceptable salt thereof.

In one embodiment, any of the formulations disclosed herein contain, unless stated otherwise, one or more pharmaceutically acceptable excipient(s) such as glidants, polymers, binders, surfactants, disintegrants, diluents, buffering agents, antiadherents, retardants, solubilizers, antioxidants, antifoaming agents, fillers, flavors, colors, lubricants, sorbents, plasticizers, or sweeteners, preservatives, or mixtures thereof, which facilitate processing of ibrutinib and/or a pharmaceutically acceptable salt thereof or into preparations which can be used pharmaceutically. Any of the well-known techniques and excipients may be used as suitable and as understood in the art, see for example, Remington: The Science and Practice of Pharmacy, Twenty-first Ed., (Pharmaceutical Press, 2005); Liberman, H. A., Lachman, L., and Schwartz, J. B. Eds., Pharmaceutical Dosage Forms, Vol. 1-2 Taylor & Francis 1990; and R. I. Mahato, Ansel's Pharmaceutical Dosage Forms and Drug Delivery Systems, Second Ed. (Taylor & Francis, 2012).

In certain embodiments, the formulations may include one or more pH adjusting agents or buffering agents, for example, acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate, ammonium chloride, and the like. The acids, bases, and buffers are added in an amount required to maintain pH of the composition in an acceptable range.

In certain embodiments, the formulations may also include one or more salts in an amount that is required to bring osmolality of the composition into an acceptable range. Such salts include those having sodium, potassium, or ammonium cations and chloride, citrate, ascorbate, borate, phosphate, bicarbonate, sulfate, thiosulfate, or bisulfite anions. Suitable salts include sodium chloride, potassium chloride, sodium thiosulfate, sodium bisulfite, and ammonium sulfate.

In certain embodiments, the formulations may also include one or more antioxidants, such as non-thiol antioxidants, e,g., ascorbic acid, butylated hydroxytoluene (BHT), butylated hydroxyanisole, sodium ascorbate, and tocopherol or derivatives thereof. In certain embodiments, antioxidants enhance chemical stability where required.

In certain embodiments, the formulations may also include one or more antifoaming agents. The foaming agent(s) are added to reduce foaming during processing which can result in coagulation of aqueous dispersions, bubbles in the finished film, or generally impair processing. Examples of suitable anti-foaming agents include silicon emulsions or sorbitan sesquoleate.

In certain embodiments, the formulations may also include one or more preservatives. Preservatives are used to inhibit microbial activity. Suitable preservatives include mercury-containing substances such as merfen and thiomersal, stabilized chlorine dioxide, and quaternary ammonium compounds such as benzalkonium chloride, cetyltrimethylammonium bromide, and cetylpyridinium chloride.

In certain embodiments, the formulations may also include one or more binders. Binders impart cohesive qualities. Exemplary binders include, e.g., alginic acid and salts thereof; cellulose derivatives, such as carboxymethylcellulose, methylcellulose (e.g., Methocel®), hydroxypropylmethylcellulose, hydroxyethylcellulose, hydroxypropylcellulose (e.g., Kiucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinyl-pyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum mucilage of isapol husks, polyvinylpyrrolidone (e.g., Polyvidone® CL, Kollidon® CL, Polyplasdone® XL-10), larch arabogalactan, Veegum®, polyethylene glycol, polyethylene oxide, waxes, sodium alginate, and the like. In general, binder levels of about 10 to about 70% are used in powder-filled gelatin capsule formulations. Binder usage level in tablet formulations varies on whether direct compression, wet granulation, or roller compaction process is used to make the tablet, and/or on types of other excipients used to make the formulation e.g, fillers which itself can act as moderate binder.

In certain embodiments, the formulations may also include dispersing agents and/or viscosity modulating agents. Dispersing agents and/or viscosity modulating agents include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween®60 or 80, PEG, polyvinylpyrrolidone (PVP; commercially known as Plasdone®), and the carbohydrate-based dispersing agents, for example, hydroxypropyl celluloses (e.g., HPC, H-PC-SL, and HPC-L), hydroxypropyl methylcelluloses (e.g., HPMC K100, RPMC K4M, HPMC K15M, and HPMC K100M), carboxymethylcellulose sodium, methylcellulose, hydroxyethyl-cellulose, hydroxypropylcellulose, hydroxypropylmethylcellulose phthalate, hydroxypropyl-methylcellulose acetate stearate (HPMCAS), noncrystalline cellulose, magnesium. aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), polyethylene oxide (e.g., PolyOx or PEO), poloxamers which are block copolymers of ethylene oxide and propylene oxide (e.g., Pluronics F68®, F88®, and F108®; and poloxamines (e.g., Tetronic 908®, also known as Poloxamine 908®, which is a block copolymer derived from sequential addition of propylene oxide and ethylene oxide to ethylenediamine (BASF Corporation, Parsippany, N.J.)), polyvinylpyrrolidone K12, K17, K25, or K30, polyvinylpyrrolidone/vinyl acetate copolymer (S-630), polyethylene glycol, e.g., the polyethylene glycol can have a molecular weight of about 300 to about 6000, or about 3350 to about 4000, or about 5400 to about 7000, polysorbate-80, sodium alginate, gums, such as, e.g., gum tragacanth and gum acacia, guar gum, xanthans, including xanthan gum, sugars, polyethoxylated sorbitan monolaurate, polyethoxylated sorbitan monolaurate, povidone, carbomers, polyvinyl alcohol (PVA), alginates, chitosans, and combinations thereof. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol, and isopropyl myristate.

In certain embodiments, the formulations may also include one or more “diluents” which refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); hydroxypropyl-methylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the formulations may also include one or more “disintegrants” which facilitate the breakup or disintegration of the dosage form when it comes in contact with the gastrointestinal fluid. Examples of disintegration agents include a starch, e.g., a natural starch such as corn starch or potato starch, a pregelatinized starch such as National 1551 or sodium starch glycolate such as Promogel®. or Explotab®, a cellulose such as a wood product, methylcrystalline cellulose, e.g., Avicel®, Avicel® PH101, Avicel® PH 102, Avicel® PH105, Elceme® P100, Emcocel®, Vivacel®, and Solka-Floc®, methylcellulose, croscarmellose, or a cross-linked cellulose, such as cross-linked sodium carboxymethyl-cellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In certain embodiments, the formulations may also include erosion facilitators which include materials that control the erosion of a particular material in gastrointestinal fluid. Exemplary erosion facilitators include, e.g., hydrophilic polymers, electrolytes, proteins, peptides, and amino acids.

In certain embodiments, the formulations may also include one or more filling agents which include compounds such as lactose, xylitol, lactitol, mannitol, sorbitol, calcium carbonate, calcium phosphate, dibasic calcium phosphate, calcium sulfate, microcrystalline cellulose, cellulose powder, dextrose, dextrates, dextran, starches, pregelatinized starch, sucrose, sodium chloride, polyethylene glycol, and the like.

In certain embodiments, the formulations may also include one or more flavoring agents and/or “sweeteners” e.g., acacia syrup, acesulfame K, alitame, anise, apple, aspartame, banana, orange, pear, peach, peppermint, peppermint cream, Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, tagatose, tangerine, thaumatin, vanilla, walnut, watermelon, wild cherry, xylitol, or any combination of thereof. these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. The flavoring agent may be incorporated with or without a polymeric coating or may be mixed directly in a formulation or first incorporated into one or more polymers.

In certain embodiments, the formulations may also include one or more plasticizers which are compounds used to soften the enteric or delayed release coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl citrate, dibutyl sebacate, triethyl cellulose, and triacetin. In some embodiments, plasticizers can also function as dispersing agents or wetting agents.

In certain embodiments, the formulations may also include one or more lubricants and glidants which are compounds that prevent, reduce or inhibit adhesion or friction of materials. Exemplary lubricants include, e.g., stearic acid, calcium hydroxide, talc, sodium stearyl lumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG4000) or a methoxypolyethylene glycol such as Carbowax®, sodium oleate, sodium benzoate, glyceryl behenate, polyethylene glycol, magnesium or sodium lauryl sulfate, colloidal silica such as Syloid®, Cab-O-Sil®, a starch such as corn starch, silicone oil, a surfactant, and the like.

In certain embodiments, the formulations may also include one or more solubilizers which include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, organic alcohols such as ethanol, n-butanol, isopropyl alcohol, hydroxypropylmethyl cellulose, hydroxypropyl beta cyclodextrins for example Captisol®, cholesterol, bile salts, propylene glycol, polyethylene glycol 200-600, glycofurol, transcutol, dimethyl isosorbide and the like. In one embodiment, the solubilizer is vitamin E TPGS and/or Captisol®. In certain embodiments, the formulations may also include one or more suspending agents which include compounds such as celluloses, such as, e.g., sodium carboxymethyl-cellulose, methylcellulose, hydroxypropylmethylcellulose, or hydroxyethylcellulose, polyvinylpyrrolidone, e.g., polyvinylpyrrolidone K112, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 5400 to about 7000, hydroxymethylcellulose acetate stearate, polysorbate-80, sodium alginate, gums, such as, e.g., gum tragacanth and gum acacia, guar gum, xanthans, including xanthan gun, sugars, polyethoxylated sorbitan monolaurate, polyethoxylated sorbitan monolaurate, povidone and the like.

In certain embodiments, the formulations may also include one or more surfactants which include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g. octoxynol 10, octoxynol 40. In some embodiments, surfactants may be included to enhance physical stability or for other purposes.

In certain embodiments, the formulations may also include one or more wetting agents which include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts, and the like.

Pharmaceutical preparations disclosed herein can be obtained by mixing one or more solid excipients such as carrier, binder, filling agent, suspending agent, flavoring agent, sweetening agent, disintegrating agent, dispersing agent, surfactant, lubricant, colorant, diluent solubilizer, moistening agent, plasticizer, stabilizer, penetration enhancer, wetting agent, anti-foaming agent, antioxidant, preservative, or one or more combinations thereof with one or more of the compounds described herein, optionally grinding the resulting mixture, and processing the mixture of granules, after adding suitable excipients, if desired, to obtain tablets.

Pharmaceutical preparations disclosed herein also include capsules made of gelatin, as well as soft, sealed capsules made of gelatin and a plasticizer, such as glycerol or sorbitol. Capsules may also be made of polymers such as hypromellose. The capsules can contain the active ingredients in admixture with filler such as lactose, binders such as starches, and/or lubricants such as talc or magnesium stearate and, optionally stabilizers. In soft capsules, the active compounds may be dissolved or suspended in suitable liquids, such as fatty oils, liquid paraffin, lipids, solubilizers, or liquid polyethylene glycols. When an enteric coated or delayed release dosage form is required, the capsule may be coated with the enteric coating or delayed release coating, or the capsule may comprise enteric coated or delayed release coated active ingredient in some form whether as directly coated, or as beads, pellets, minitabs, or another sutiable form.

These formulations can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, (6) fusion, or (7) extrusion. See, e.g., Lachman et al., The Theory and Practice of Industrial Pharmacy, 3^{rd} ed. (1986). Other methods include, e.g., spray drying, pan coating, melt granulation, granulation, fluidized bed spray drying or coating (e.g., wurster coating), high shear granulation, tangential coating, top spraying, tableting, extruding, extrusion/spheronization, and the like.

It should be appreciated that there is considerable overlap between excipients used in the solid dosage forms described herein. Thus, the above-listed additives should be taken as merely exemplary, and not limiting, of the types of excipients that can be included in solid dosage forms described herein. The type and amounts of such excipients can be readily determined by one skilled in the art, according to the particular properties desired.

In some embodiments, the solid dosage forms described herein are enteric coated oral dosage forms, i.e., as an oral dosage form of a pharmaceutical composition as described herein which utilizes an enteric coating to effect the release of the compound in the intestine of the gastrointestinal tract rather than in the stomach. An “enterically coated” drug or tablet refers to a drug or tablet that is coated with a substance—i.e., with an “enteric coating”—that remains intact in the stomach but dissolves and releases the drug once the intestine is reached. As used herein “enteric coating”, is a material, such as a polymer material or materials which encase the therapeutically active agent core either as a dosage form or as particles. Typically, a substantial amount or all of the enteric coating material is dissolved before the therapeutically active agent is released from the dosage form, so as to achieve delayed dissolution of the therapeutically active agent core or particles in the intestine. Enteric coatings are discussed, for example, Loyd, V. Allen, Remington: The Science and Practice of Pharmacy, Twenty-first Ed., (Pharmaceutical Press, 2005; and P. J. Tarcha, Polymers for Controlled Drug Delivery, Chapter 3, CRC Press, 1991. Methods for applying enteric coatings to pharmaceutical compositions are well known in the art, and include for example, U.S. Patent Application Publication No. 2006/0045822.

The enteric coated dosage form may be a compressed or molded or extruded tablet (coated or uncoated) containing granules, powder, pellets, beads or particles of ibrutinib and/or a pharmaceutically acceptable salt thereof and/or other excipients, which are themselves coated or uncoated provided at least either the dosage form or ibrutinib and/or a pharmaceutically acceptable salt thereof is coated. The enteric coated oral dosage form may also be a capsule (coated or uncoated) containing pellets, beads or granules of ibrutinib and/or a pharmaceutically acceptable salt thereof or and/or other excipients, which are themselves coated or uncoated provided at least either the dosage form or ibrutinib and/or a pharmaceutically acceptable salt thereof is coated. Some examples of coatings that were originally used as enteric coatings are beeswax and glyceryl monostearate; beeswax, shellac and cellulose; and cetyl alcohol, mastic and shellac as well as shellac and stearic acid (see U.S. Pat. No. 2,809,918); polyvinylacetate and ethyl cellulose (see U.S. Pat. No. 3,835,221). More recently, the coatings used are neutral copolymers of polymethacrylic acid esters (Eudragit L30D). (F. W. Goodhart et al, Pharm. Tech., p. 64-71, April, 1984); copolymers of methacrylic acid and methacrylic acid methyl ester (Eudragit S), or a neutral copolymer of polymethacrylic acid esters containing metallic stearates (Mehta et. al. U.S. Pat. Nos. 4,728,512 and 4,794,001), cellulose acetate succinate, and hypromellose phthalate.

Any anionic polymer exhibiting a pH-dependent solubility profile can be used as an enteric coating in the methods and compositions described herein to achieve delivery to the intestine, such as the small intestine, for example the duodenum and/or jejunum. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH>7;

Acrylic polymers: The performance of acrylic polymers (primarily their solubility in biological fluids) can vary based on the degree and type of substitution. Examples of suitable acrylic polymers include methacrylic acid copolymers and ammonium methacrylate copolymers. The Eudragit series L, S, and RS (manufactured Rohm Pharma and known as Evonik®) are available as solubilized in organic solvent, aqueous dispersion, or dry powders. The Eudragit series RL, NE, and RS are insoluble in the gastrointestinal tract but are permeable and are used primarily for colonic targeting. The Eudragit series L, L-30D and S are insoluble in stomach and dissolve in the intestine;

Cellulose Derivatives: Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP pseudolatex with particles <1 Other components in Aquateric can include Pluronics, Tweens, and acetylated monoglycerides. Other suitable cellulose derivatives include; cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP): PVAP dissolves at pH>5, and it is much less permeable to water vapor and gastric fluids. Detailed description of above polymers and their pH-dependent solubility can be found at in the article titled “Enteric coated hard gelatin capsules” by Professor Karl Thoma and Karoline Bechtold at http://pop.www.capsugel.com/media/library/enteric-coated-hard-gelatin-capsules.pdf

In one embodiment the enteric coating is made from methacrylic acid copolymers; cellulose acetate (and its succinate and phthalate version), polymethacrylic acid/acrylic acid copolymer, hydroxypropyl methyl cellulose phthalate, polyvinyl acetate phthalate, hydroxyethyl ethyl cellulose phthalate, cellulose acetate tetrahydrophtalate, acrylic resin or shellac. In another embodiment the polymer is chosen from cellulose acetate phthalate (CAP; dissolves above pH 6), polyvinyl acetate phthalate (PVAP, disintegrates at pH 5), hydroxypropyl methyl cellulose pthalate (HPMCP, grade HP50 disintegrates at pH 5 and HP50 disintegrates at 5.5), methylacrylic acid copolymers (Eudragit L and S, Eudragit L disintegrates at pH 6 and Eudragit S disintegrates at pH 7). In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as fluid bed or Wurster coaters, or spray or pan coating are employed to apply coatings. The coating thickness must be sufficient to ensure that the oral dosage form remains intact until the desired site of topical delivery in the intestinal tract is reached, and screening of coatings of varying thickness in dissolution testing at acidic and near neutral pH is well known in the art for selecting the appropriate coating and thickness.

Colorants, surfactants, anti-adhesion agents, antifoaming agents, lubricants (e.g., carnuba wax or PEG) and other additives may be added to the coatings besides plasticizers to solubilize or disperse the coating material, and to improve coating performance and the coated product.

To accelerate the dissolution of the enteric coat, a half-thickness, double coat of enteric polymer (for instance, Eudragit L30 D-55) may be applied, and the inner enteric coat may have a buffer up to pH 6.0 in the presence of 10% citric acid, followed by a final layer of standard Eudragit L 30 D-55. Applying two layers of enteric coat, each half the thickness of a typical enteric coat, Liu and Basit were able to accelerate enteric coating dissolution compared to a similar coating system applied, unbuffered, as a single layer (Liu, F. and Basit, A. Journal of Controlled Release. 147 (2010) 242-245.)

The intactness of the enteric coating may be measured, for example, by the degradation of the drug within the micropellets. The enteric coated doage forms or pellets may be tested in dissolution testing first in gastric fluid and separately in intestinal fluid as described in USP to determine its function.

The enteric coated tablets and capsules formulation containing the disclosed compounds can be made by methods well known in the art. For example, tablets containing a compound disclosed herein can be enterically coated with a coating solution containing Eudragit®, diethylphthlate, isopropyl alcohol, talc, and water using a side vented coating pan (Freund Hi-Coater).

Alternatively, a multi-unit dosage form comprising enteric-coated pellets that can be incorporated into a tablet or into a capsule can be prepared as follows.

Core material: The core material for the individually enteric coating layered pellets can be constituted according to different principles. Seeds layered with the active agent, optionally mixed with alkaline substances or buffer, can be used as the core material for the further processing.

The seeds which are to be layered with the active agent can be water insoluble seeds comprising different oxides, celluloses, organic polymers and other materials, alone or in mixtures or water-soluble seeds comprising different inorganic salts, sugars, non-pareils and other materials, alone or in mixtures. Further, the seeds may comprise the active agent in the form of crystals, agglomerates, compacts etc. The size of the seeds is not essential for the present disclosure but may vary between approximately 0.1 and 4 mm, such as less than 2 mm. The seeds layered with the active agent are produced either by powder or solution/suspension layering using for instance granulation or spray coating layering equipment.

Before the seeds are layered, active agent may be mixed with further components. Such components can be binders, surfactants fillers, disintegrating agents, alkaline additives or other and/or pharmaceutically acceptable ingredients alone or in mixtures. The binders are for example polymers such as hydroxypropyl methylcellulose (HPMC), hydroxypropyl-cellulose (HPC), carboxymethylcellulose sodium, polyvinyl pyrrolidone (PVP), or sugars, starches or other pharmaceutically acceptable substances with cohesive properties. Suitable surfactants are found in the groups of pharmaceutically acceptable non-ionic or ionic surfactants or combinations thereof such as for instance sodium lauryl sulfate or Tween 80.

Alternatively, the active agent optionally mixed with with suitable constituents can be formulated into a core material. Said core material may be produced by extrusion/spheronization, balling or compression utilizing conventional process equipment. The size of the formulated core material is approximately between 0.1 and 4 mm, such as between 0.1 and 2 mm. The manufactured core material can further be layered with additional ingredients comprising the active agent and/or be used for further processing.

The active agent is mixed with pharmaceutical constituents to obtain preferred handling and processing properties and a suitable concentration of the active agent in the final preparation. Pharmaceutical constituents such as fillers, binders, lubricants, disintegrating agents, surfactants, and other pharmaceutically acceptable additives may be used.

Alternatively, the aforementioned core material can be prepared by using spray drying or spray congealing technique.

Enteric Coating Layer(s): Before applying the enteric coating layer(s) onto the core material in the form of individual pellets, the pellets may optionally be covered with one or more separating layer(s) comprising pharmaceutical excipients optionally including alkaline compounds such as pH-buffering compounds. This/these separating layer(s), separate(s) the core material from the outer layers being enteric coating layer(s). This/these separating layer(s) protecting the core material of active agent should be water soluble or rapidly disintegrating in water.

A separating layer(s) can be optionally applied to the core material by coating or layering procedures in suitable equipments such as coating pan, coating granulator or in a fluidized bed apparatus using water and/or organic solvents for the coating process. As an alternative the separating layer(s) can be applied to the core material by using powder coating technique. The materials for the separating layers are pharmaceutically acceptable compounds such as, for instance, sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium, water soluble salts of enteric coating polymers and others, used alone or in mixtures. Additives such as plasticizers, colorants, pigments, fillers anti-tacking and anti-static agents, such as for instance magnesium stearate, titanium dioxide, talc and other additives may also be included into the separating layer(s).

When the optional separating layer, is applied to the core material it may constitute a variable thickness. The maximum thickness of the separating layer(s) is normally only limited by processing conditions. The separating layer may serve as a diffusion barrier and may act as a pH-buffering zone. The optionally applied separating layer(s) is not essential. However, the separating layer(s) may improve the chemical stability of the active substance and/or the physical properties of the novel multiple unit tableted dosage form.

Alternatively, the separating layer may be formed in situ by a reaction between an enteric coating polymer layer applied on the core material and an alkaline reacting compound in the core material. Thus, the separating layer formed comprises a water soluble salt formed between the enteric coating layer polymer(s) and an alkaline reacting compound which is in the position to form a salt

One or more enteric coating layers are applied onto the core material or onto the core material covered with separating layer(s) by using a suitable coating technique. The enteric coating layer material may be dispersed or dissolved in either water or in suitable organic solvents. As enteric coating layer polymers one or more, separately or in combination, of the following can be used, e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, or other suitable enteric coating polymer(s).

The enteric coating layers contain pharmaceutically acceptable plasticizers to obtain the desired mechanical properties, such as flexibility and hardness of the enteric coating layers. Such plasticizers are for instance, but not restricted to triacetin, citric acid esters, phthalic acid esters, dibutyl sebacate, cetyl alcohol, polyethylene glycols, polysorbates or other plasticizers.

The amount of plasticizer is optimized for each enteric coating layer formula, in relation to the selected enteric coating layer polymer(s), selected plasticizer(s) and the applied amount of said polymer(s), in such a way that the mechanical properties, i.e. flexibility and hardness of the enteric coating layer(s), for instance exemplified as Vickers hardness, are adjusted so that if a tablet is desired the acid resistance of the pellets covered with enteric coating layer(s) does not decrease significantly during compression of pellets into tablets. The amount of plasticizer is usually above 5% by weight of the enteric coating layer polymer(s), (In one embodiment the amount of plasticizer is 15-50%. In another embodiment the amount of plasticizer is 20-50%). Additives such as dispersants, colorants, pigments polymers e.g. poly(ethylacrylate, methylmethacrylate), anti-tacking and anti-foaming agents may also be included into the enteric coating layer(s). Other compounds may be added to increase film thickness and to decrease diffusion of acidic gastric juices into the acid susceptible material. The maximum thickness of the applied enteric coating is normally only limited by processing conditions and the desired dissolution profile.

Over-Coating Layer: Pellets covered with enteric coating layer(s) may optionally further be covered with one or more over-coating layer(s). The over-coating layer(s) should be water soluble or rapidly disintegrating in water. The over-coating layer(s) can be applied to the enteric coating layered pellets by coating or layering procedures in suitable equipments such as coating pan, coating granulator or in a fluidized bed apparatus using water and/or organic solvents for the coating or layering process. The materials for over-coating layers are chosen among pharmaceutically acceptable compounds such as, for instance sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium and others, used alone or in mixtures. Additives such as plasticizers, colorants, pigments, fillers, anti-tacking and anti-static agents, such for instance magnesium stearate, titanium dioxide, talc and other additives may also be included into the over-coating layer(s). The over-coating layer may further prevent potential agglomeration of enteric coating layered pellets, further it may protect the enteric coating layer towards cracking during the compaction process and enhance the tableting process. The maximum thickness of the applied over-coating layer(s) is normally limited by processing conditions and the desired dissolution profile. The over-coating layer may also be used as a tablet film coating layer.

Enteric coating of soft gelatin capsules may contain an emulsion, oil, microemulsion, self-emulsifying system, lipid, triglycerides, polyethylene glycol, surfactants, other solubilizers and the like, and combinations thereof, to solubilize the active agent. The flexibility of the soft gelatin capsule is maintained by residual water and plasticizer. Moreover, for gelatin capsules the gelatin may be dissolved in water so that spraying must be accomplished at a rate with relatively low relative humidity such as can be accomplished in a fluid bed or Wurster. In addition, drying must be accomplished without removing the residual water or plasticizer causing cracking of the capsule shell. Commercially available blends optimized for enteric coating of soft gelatin capsules such as Instamodel EPD (Enteric Polymeric Dispersion), available from Ideal Cures, Pvt. Ltd. (Mumbai, India). On a laboratory scale enteric coated capsules may be prepared by: a) rotating capsules in a flask or dipping capsules in a solution of the gently heated enteric coating material with plasticizer at the lowest possible temperature or b) in a lab scale sprayer/fluid bed and then drying.

For aqueous active agents, it can be especially desirable to incorporate the drug in the water phase of an emulsion. Such “water-in-oil” emulsion provide a suitable biophysical environment for the drug and can provide an oil-water interface that can protect the drug from adverse effects of pH or enzymes that can degrade the drug. Additionally, such water-in-oil formulations can provide a lipid layer, which can interact favorably with lipids in cells of the body, and can increase the partition of the formulation into the membranes of cells. Such partition can increase the absorption of drugs in such formulations into the circulation and therefore can increase the bioavailability of the drug.

In some embodiments the water-in-oil emulsion contains an oily phase composed of long chain carboxylic acids or esters or alcohols thereof, a surfactant or a surface active agent, and an aqueous phase containing primarily water and the active agent.

Long chain carboxylic acids are those ranging from C₈ to C₂₂ with up to three unsaturated bonds (also branching). Examples of saturated straight chain acids are n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. Also useful are unsaturated monoolefinic straight chain monocarboxylic acids. Examples of these are oleic acid, gadoleic acid and erucic acid. Also useful are unsaturated (polyolefinic) straight chain monocarboxylic acids. Examples of these are linoleic acid, ricinoleic acid, linolenic acid, arachidonic acid and behenolic acid. Useful branched acids include, for example, diacetyl tartaric acid.

Examples of long chain carboxylic acid esters include, but are not limited to, those from the group of: glyceryl monostearates; glyceryl monopalmitates; mixtures of glyceryl monostearate and glyceryl monopalmitate; glyceryl monolinoleate; glyceryl monooleate; mixtures of glyceryl monopalmitate, glyceryl monostearate, glyceryl monooleate and glyceryl monolinoleate; glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate; acetylated glycerides such as distilled acetylated monoglycerides; mixtures of propylene glycol monoesters, distilled monoglycerides, sodium steroyl lactylate and silicon dioxide; d-alpha tocopherol polyethylene glycol 1000 succinate; mixtures of mono- and di-glyceride esters such as Atmul; calcium stearoyl lactylate; ethoxylated mono- and di-glycerides; lactated mono- and di-glycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; stearyl octanoate; C₈-C₃₀ cholesterol/lavosterol esters; and sucrose long chain carboxylic acid esters. Examples of the self-emulsifying long chain carboxylic acid esters include those from the groups of stearates, pamitates, ricinoleates, oleates, behenates, ricinolenates, myristates, laurates, caprylates, and caproates. In some embodiments the oily phase may comprise a combination of 2 or more of the long chain carboxylic acids or esters or alcohols thereof. In some embodiments the oil phase may comprise a mixture of caprylic/capric triglyceride and C₈/C₁₀ mono-/di-glycerides of caprylic acid.

The alcohols that can be used are exemplified by the hydroxyl forms of the carboxylic acids exemplified above and also strearyl alcohol.

Surface active agents or surfactants are long chain molecules that can accumulate at hydrophilic/hydrophobic (water/oil) interfaces and lower the surface tension at the interface. As a result they can stabilise an emulsion. In some embodiments, the surfactant may comprise: Tween® (polyoxyethylene sorbate) family of surfactants, Span® (sorbitan long chain carboxylic acid esters) family of surfactants, Pluronic® (ethylene or propylene oxide block copolymers) family of surfactants, Labrasol®, Labrafil® and Labrafac®(each polyglycolyzed glycerides) families of surfactants, sorbitan esters of oleate, stearate, laurate or other long chain carboxylic acids, poloxamers (polyethylene-polypropylene glycol block copolymers or Pluronic®), other sorbitan or sucrose long chain carboxylic acid esters, mono and diglycerides, PEG derivatives of caprylic/capric triglycerides and mixtures thereof or mixture of two or more of the above. In some embodiments the surfactant phase may comprise a mixture of Polyoxyethylene (20) sorbitan monooleate (Tween 80®) and sorbitan monooleate (Span 80®).

The aqueous phase may optionally comprise the active agent suspended in water and a buffer.

In some embodiments, such emulsions are coarse emulsions, microemulsions and liquid crystal emulsions. In other embodiments such emulsion may optionally comprise a permeation enhancer. In other embodiments, spray-dried dispersions or microparticles or nanoparticles containing encapsulated microemulsion, coarse emulsion or liquid crystal can be used.

In some embodiments, the solid dosage forms described herein are non-enteric time-delayed release dosage forms. The term “non-enteric time-delayed release” as used herein refers to the delivery so that the release of the drug can be accomplished at some generally predictable location in the intestinal tract more distal to that which would have been accomplished if there had been no delayed release alterations. In some embodiments the method for delay of release is a coating that becomes permeable, dissolves, ruptures, and/or is no longer intact after a designed duration.

The coating in the time-delayed release dosage forms can have a fixed time to erode after which the drug is released (suitable coating include polymeric coating such as HPMC, and the like) or has a core comprised of a disinegrant(s) or osmotic agent(s) such as a salt, hydrophilic polymer, typically polyethylene oxide or an alkylcellulose, sugar, or the like, which draw(s) water through a membrane or a gas generating agent such as citric acid and sodium bicarbonate. The membrane may rupture after the swelling pressure exceeds a certain threshold over a desired delay time. Alternatively, a membrane could become porous by leaching an aqueous extractable over a desired delay time. The time delayed dosage forms are sometimesadministered in a fasted state to avoid variability in gastric emptying in the fed state.

The time-delayed dosage form can be in the form of a mechanical dosage form, e.g., as a tablet or capsule, such as an Enterion® capsule or Heidelberg® capsule (pH sensitive) which can release the drug when it receives a signal which can be transmitted once it leaves the stomach.

In a fifth aspect, the present disclosure is directed to a method of increasing bioavailability of ibrutinib and/or a pharmaceutically acceptable salt thereof in a patient, which method comprises administering ibrutinib and/or a pharmaceutically acceptable salt thereof to the patient in a solid oral dosage form that releases said ibrutinib and/or a pharmaceutically acceptable salt thereof in the intestine. In one embodiment, the bioavailability is increased by administering ibrutinib and/or a pharmaceutically acceptable salt thereof to the patient in any of the solid oral dosage forms disclosed herein.

In a sixth aspect, the present disclosure is directed method of treating a disease treatable by inhibition of a tyrosine kinase in a patient in recognized need thereof which method comprises administering to said patient, in single or multiple doses, a therapeutically effective amount of ibrutinib and/or a pharmaceutically acceptable salt thereof in a solid oral dosage form that releases said ibrutinib and/or said pharmaceutically acceptable salt thereof in the intestine. The present disclosure is also directed method of treating a disease treatable by inhibition of a tyrosine kinase in a patient in recognized need thereof which method comprises administering to said patient, in single or multiple doses, any of the solid oral dosage forms disclosed herein.

In a seventh aspect, the present disclosure is directed method of treating a disease treatable by inhibition of a tyrosine kinase in a patient in recognized need thereof which method comprises administering to said patient, in single or multiple doses, a therapeutically effective amount of ibrutinib and/or a pharmaceutically acceptable salt thereof in a solid oral dosage form disclosed herein. In one embodiment of sixth and seventh aspects, the tyrosine kinase is chosen from BLK, BMX, EGFR, HER2, HER4, ITK, TEC, BTK, and TXK. In another embodiment of sixth and seventh aspects, the tyrosine kinase is BTK. In another embodiment of sixth aspect and embodiments contained therein said ibrutinib and/or said pharmaceutically acceptable salt thereof is released in the small intestine. In another embodiment of sixth and seventh aspects and embodiments contained therein said therapeutically effective amount of said ibrutinib and/or said pharmaceutically acceptable salt thereof is from about 20 mg/day to about 450 mg/day.

In another embodiment of the sixth and seventh aspects and embodiments contained therein said therapeutically effective amount of said ibrutinib and/or said pharmaceutically acceptable salt thereof is from about 30 mg/day to about 300 mg/day. In another embodiment of the sixth and seventh aspects and embodiments contained therein said therapeutically effective amount of said ibrutinib and/or said pharmaceutically acceptable salt thereof is from about 50 mg/day to about 220 mg/day. In another embodiment of the sixth and seventh aspects and embodiments contained therein, the dosage form comprises at least one coating chosen from enteric coatings and non-enteric time-delayed release coatings; and at least one pharmaceutically acceptable excipient. In another embodiment of the sixth and seventh aspects and embodiments contained therein, the disease is cancer or inflammatory disease. In another embodiment of the sixth and seventh aspects and embodiments contained therein the disease is leukemia. In another embodiment of the sixth and seventh aspects and embodiments contained therein the disease is leukemia chosen from chronic lymphocytic leukemia (CLL), small lymphocytic lymphoma (SLL), and B-cell non-Hodgkin lymphoma. In another embodiment of the sixth and seventh aspects and embodiments contained therein, said ibrutinib and/or at least one pharmacuetically acceptable salt thereof, is administered in combination with at least one additional agent chosen from anti-inflammatory and antiproliferative agents such as ofatumumab, bendamustine, and rituaximab.

In an eighth aspect, the present disclosure is directed to a method of treating cancer or an autoimmune disease in a patient comprising administering to the patient in recognized need thereof, a solid oral dosage form disclosed herein.

In one embodiment the disease is inflammatory disease such as arthritis, kidney disease, or cancer, such as leukemia, for example chronic lymphocytic leukemia (CLL), multiple myeloma, and small lymphocytic lymphoma (SLL), and B-cell non-Hodgkin lymphoma.

In one embodiment of this aspect, the subject in need is suffering from an autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia. In some embodiments, the disease is rheumatoid arthritis. In some embodiments, the autoimmune disease is lupus. In another embodiment of this aspect, the patient in need is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In another embodiment of this aspect, the patient in need is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis. In another embodiment of this aspect, the patient is suffering from inflammatory skin disease which includes, by way of example, dermatitis, contact dermatitis, eczema, urticaria, rosacea, and scarring psoriatic lesions in the skin, joints, or other tissues or organs.

In yet another embodiment of this aspect, the subject in need is suffering from a cancer. In one embodiment, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, the oral formulation of the present disclosure (or any of the embodiments thereof described herein), is administered in combination with another an anti-cancer agent e.g., the anti-cancer agent is an inhibitor of mitogen-activated protein kinase signaling, e.g., U0126, PD98059, PD184352, PD0325901, ARRY-142886, SB239063, SP600125, BAY 43-9006, wortmannin, Nexavar®, Tarceva®, Sutent®, Tykerb®, Sprycel®, Crizotinib, Xalkori®, LY294002, ofatumumab, bendamustine, or rituaximab.

In yet another embodiment, the patient in need is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

A ninth aspect is the use of a solid oral dosage form comprising an enteric coated ibrutinib and/or a pharmaceutically acceptable salt thereof; and further comprising at least one pharmaceutically acceptable excipient for treating an inflammatory disease or proliferative disease in a patient in which the activity of a tyrosine kinase such as BLK, BMX, EGFR, HER2, HER4, ITK, TEC, BTK, and TXK, in particular, BTK contributes to the pathology and/or symptoms of the disease. In one embodiment of this aspect, the tyrosine kinase protein is BTK.

In any of the aforementioned aspects involving the treatment of proliferative disorders, including cancer, are further embodiments comprising administering ibrutinib and/or a pharmacuetically acceptable salt thereof, in combination with at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzamab, methotrexate, paclitaxel, Taxol™, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol. When combination therapy is used, the agents can be administered simultaneously or sequentially.

### DEFINITIONS

Unless otherwise stated, the following terms used in the specification and claims are defined for the purposes of this Application and have the following meaning:

“Active compound, agent, substance, ingredient, or drug” means ibrutinib or a pharmaceutically acceptable salt thereof, unless stated otherwise.

“Enteric coating or non-enteric time delayed coating” as used herein means a coating that prevents delivery/release of the active compound to the stomach and allows release in the intestine and is tested to resist release of drug at acidic pH and to release drug at pH greater than or equal to 5. In one embodiment the drug is released at at least pH greater than or equal to 5.5.

The present disclosure also includes the prodrugs of compounds of ibrutinib or a pharmaceutically acceptable salt thereof. The term prodrug is intended to represent covalently bonded carriers, which are capable of releasing ibrutinib, when the prodrug is administered to a mammalian subject. Release of the active ingredient occurs in vivo. Prodrugs can be prepared by techniques known to one skilled in the art. These techniques generally modify appropriate functional groups in a given compound.

The present disclosure also includes polymorphic forms (amorphous as well as crystalline) and deuterated forms of compounds of ibrutinib or pharmaceutically acceptable salt thereof.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present disclosure may have asymmetric centers. Compounds of the present disclosure containing an asymmetrically substituted atom may be isolated in optically active or racemic forms. It is well known in the art how to prepare optically active forms, such as by resolution of materials. All chiral, diastereomeric, racemic forms are within the scope of this disclosure, unless the specific stereochemistry or isomeric form is specifically indicated.

“Oral bioavailability” refers to the extent to and rate at which the active moiety (drug or metabolite) enters systemic circulation when the drug is administered orally versus when the drug is administered intravenuosly, the bioavailability of the drug being 100% when it is adminstered intravenously. Methods to determine the bioavailability of drugs are well known to those of ordinary skill in the art e.g., area under the plasma concentration-time curve (AUC).

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Release in the intestine” means that greater than 50% of ibrutinib and/or a pharmaceutically acceptable salt thereof is released from the dosage form directly in the intestine ie., bypasses exposure to the stomach. In one embodiment, about 60%, 70%, 75%, 80%, 85%, 90%, 95%, or 100% of ibrutinib and/or a pharmaceutically acceptable salt thereof is directly released from the dosage form in the intestine. In another embodiment, about 80%, 85%, 90%, 95%, or 100% of ibrutinib and/or a pharmaceutically acceptable salt thereof is directly released from the dosage form in the intestine. In another embodiment, about 100% of ibrutinib and/or a pharmaceutically acceptable salt thereof is directly released from the dosage from in the intestine.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

A “therapeutically effective amount” means the amount of ibrutinib and/or a pharmaceutically acceptable salt thereof that, when administered to a mammal for treating a disease, is sufficient to effect such treatment for the disease. The “therapeutically effective amount” will vary depending on the compound, the disease and its severity and the age, weight, etc., of the mammal to be treated. The therapeutically effective amount of ibrutinib and/or a pharmaceutically acceptable salt thereof when administered in the intestine can be from about 20 mg per day to about 450 mg/day, or any permuations and combinations thereof, such as 20 mg/day to about 420 mg/day; or about 20 mg/day or 30 mg/day to about 300 or 350 mg/day; or about 30 or 50 mg/day to about 200, or 220 or 250 mg/day; or about 30 or 50 mg/day to about 100 or 150 mg/day and can be administered in single or multiple doses.

The compounds of the present disclosure may be used in combination with one or more other drugs in the treatment of diseases or conditions for which compounds of the present disclosure or the other drugs may have utility, where the combination of the drugs together are safer or more effective than either drug alone. Such other drug(s) may be administered, by a route and in an amount commonly used therefore, contemporaneously or sequentially with a compound of the present disclosure. When a compound of the present disclosure is used contemporaneously with one or more other drugs, a pharmaceutical composition in unit dosage form containing such other drugs and the compound of the present disclosure is preferred. However, the combination therapy may also include therapies in which the compound of the present disclosure and one or more other drugs are administered on different overlapping schedules. It is also contemplated that when used in combination with one or more other active ingredients, the compounds of the present disclosure and the other active ingredients may be used in lower doses than when each is used singly.

Accordingly, the pharmaceutical compositions of the present disclosure also include those that contain one or more other active ingredients, in addition to a compound of the present disclosure.

The above combinations include combinations of a compound of the present disclosure not only with one other active compound, but also with two or more other active compounds. Likewise, compounds of the present disclosure may be used in combination with other drugs that are used in the prevention, treatment, control, amelioration, or reduction of risk of the diseases or conditions for which compounds of the present disclosure are useful. Such other drugs may be administered, by a route and in an amount commonly used therefore, contemporaneously or sequentially with a compound of the present disclosure. When a compound of the present disclosure is used contemporaneously with one or more other drugs, a pharmaceutical composition containing such other drugs in addition to the compound of the present disclosure is preferred. Accordingly, the pharmaceutical compositions of the present disclosure also include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used.

Where the subject is suffering from or at risk of suffering from an autoimmune disease, an inflammatory disease, or an allergy disease, the pharmaceutical compositions of the present disclosure can be used in with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-.alpha. binding proteins (e.g., infliximab, etanercept, or adalimumab), abatacept, anakinra, interferon-.beta., interferon-.gamma., interleukin-2, allergy vaccines, antihistamines, antileukotrienes, beta-agonists, theophylline, or anticholinergics.

Where the subject is suffering from or at risk of suffering from a B-cell proliferative disorder (e.g., plasma cell myeloma), the subject can be treated with the pharmaceutical compositions of the present disclosure in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec™) geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™., such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with the pharmaceutical compositions of the present disclosure, include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents that can be employed in combination with the pharmaceutical compositions of the present disclosure) include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-nl; interferon alfa-n3; interferon beta-1a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with the pharmaceutical compositions of the present disclosure include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with the pharmaceutical compositions of the present disclosure include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products useful in combination with the pharmaceutical compositions of the present disclosure include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination with the pharmaceutical compositions of the present disclosure include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, Cytarabine), purine analogs (e.g., mercaptopurine, thioguanine, pentostatin.

Examples of hormones and antagonists useful in combination with the pharmaceutical compositions of the present disclosure include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an BTK inhibitor compound of the disclosure include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Where the subject is suffering from or at risk of suffering from a thromboembolic disorder (e.g., stroke), the subject can be treated with the pharmaceutical compositions of the present disclosure in any combination with one or more other anti-thromboembolic agents. Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIER 1048.

### FORMULATION EXAMPLES

### Example 1

### Ibrutinib and/or a Pharmaceutically Acceptable Salt Thereof Dissolved in Lipids Contained in Enteric-Coated Hard Gelatin Capsules

24 G of ibrutinib and/or a pharmaceutically acceptable salt thereof is dissolved as a 12% (w/w) solution of glyceryl tricaprylate/tricaprate (Captex® 355) by adding first to a 0.5 kg glass mixing vessel, 88 g of Captex, followed by 24 g of ibrutinib and/or a pharmaceutically acceptable salt thereof and gentle stirring for 5 minutes, and then followed by the remaining 88 g of the Captex and stirring is continued until dissolved. 200 mg of ibrutinib and/or a pharmaceutically acceptable salt thereof solution is dispensed into each of 900 hard gelatin capsules (size 1). The Coni-Snap hard gelatin Licap capsules (Capsugel) cap and body joints are then sprayed with 50% aqueous ethanol for about 1 second to lower the sealing temperature of the gelatin. The capsules are then sealed by heating the joint to 55° C. for about 1 minute.

The filled gelatin capsules are placed in a fluid-bed coater to apply the enteric coating. The coating solution is 82.89% Eudragit L30D mixed with 10% aqueous solution of PEG 6000 (8.29% w/w), talc (8.29% w/w), and 0.51% simethicone. A peristaltic pump (6-10 rpm) is used to deliver the spraying solution to the nozzle. The dispersion is slowly stirred. The fluid bed is operated with inlet and outlet temperatures, respectively of 55 and 45° C., with an atomization pressure of 1.2-1.5 kg/cm², and an exhaust air velocity of 42 ft³/min. The enteric-coated capsules are sprayed until 5% weight gain, and then dried at 45° C. The capsules are packaged in capped and sealed HDPE bottles and stored at ambient temperature.

### Example 2

### Ibrutinib and/or a Pharmaceutically Acceptable Salt Thereof in Non-Enteric, Delayed Time Released Tablet Made by Coating

To make immediate release tablet cores of ibrutinib and/or a pharmaceutically acceptable salt thereof, a high shear granulation is prepared by blending 10 kg of ibrutinib and/or a pharmaceutically acceptable salt thereof, 1 kg microcrystalline cellulose or lactose or a combination of the two excipients, and 900 g of starch in a granulating bowl. After forming the wet mass by granulating with water, the granules are dried in a fluid bed dryer until the water content is less than 3%. After milling the dried granulation, it is sieved through 16 to 20 mesh. This granulate is then blended with 400 g sodium starch glycolate, 40 g magnesium stearate and 20 g silicon dioxide. This powder blend is then tableted using conventional tablet press equipped with B tooling to give a tablet weight between 100 and 600 mg.

To prepare a delayed release coating on these tablets, a coating solution is made comprised of 35% Eudragit RS 30D, 4.97% Eudragit RL 30D, 7% talc, 8% triethylcitrate, 0.03% simethicone, and 45% water. The talc and water are charged in a stainless steel vessel, and then mixed slowly until suspended. In a second stainless vessel, the Eudragit RS 30D and the simethicone are mixed followed by the addition of the Eudragit RL 30D and the plasticizer. Finally, the talc suspension is added to form the coating solution. The tablets are then placed in a fluid bed coater and coated with about half to 1.5 times the weight of the tablets to the desired weight gain. Talc may be added to dust the tablets during the coating. After coating, the coated tablets are dried.

### Example 3

### Ibrutinib and/or a Pharmaceutically Acceptable Salt Thereof in Non-Enteric Delayed Time Released Tablet Made by Press Coating

As in example 2, to make immediate release tablet cores of ibrutinib and/or a pharmaceutically acceptable salt thereof, a high shear granulation is prepared by blending 10 kg of ibrutinib and/or a pharmaceutically acceptable salt thereof, 1 kg microcrystalline cellulose or lactose or a combination of the two excipients, and 900 g of starch in a granulating bowl. After forming the wet mass by granulating with water, the granules are dried in a fluid bed dryer until the water content is less than 3%. After milling the dried granulation, it is sieved through 16 to 20 mesh. This granulate is then blended in a small V-blender with 400 g sodium starch glycolate, 40 g magnesium stearate and 20 g silicon dioxide. This powder blend is then tableted using a Manesty Dry-Cota tablet press with a flat face, round 0.2031″ die and punch. The tablet hardness is controlled to 4±2 kp.

To apply the press coating, 2 kg hypromellose 2208 and 2 kg microcrystalline cellulose are blended in a V-blender for 10 minutes. 0.04 kg of magnesium stearate is then added and mixed for another 10 minutes. The cores from above are then press coated in a Dry-Cota press with a 0.3600″ round shallow, concave punch and die.

### Example 4

### Ibrutinib and/or a Pharmaceutically Acceptable Salt Thereof in Enteric Coated Beads

1 kg of ibrutinib and/or a pharmaceutically acceptable salt thereof and 0.1 kg talc are blended for 15 minutes in a V-blender. Then milled and screened to yield a fine powder. A binder solution is prepared with 10% (w/v) PVP in water. A coating pan is then charged with 1 kg of inert sugar spheres (20 to 50 mesh). The sugar spheres are then sprayed with the binder solution and the drug blend is applied to the spheres until all ibrutinib and/or a pharmaceutically acceptable salt thereof is consumed. The drug-loaded beads are then dried in a fluid bed dryer.

A fluidized bed coater is loaded with 1 kg of the above drug-loaded beads. The beads are then coated with 1 kg of the coating solution from Example 1, and then dried. Talc may be used to reduce tackiness during the coating process.

### Example 5

### Determination of Bioavailability Stomach v. Intraduodenal Adminstration

Fed female Sprague dawley rats, 225-250 g with surgically implanted intra-duodenal catheters were obtained commercially. Rats were dosed solutions of ibrutinib via bolus injection: oral gavage was done to measure exposure after oral dosing, or dosing via administration through the intra-duodenal catheter to measure exposure bypassing the stomach. Ibrutinib was dosed at 20 mg/kg at a dose volume of 2 ml/kg. Blood was withdrawn at time points out to 24 hours and Ibrutinib quantitated via LC/MS/MS to obtain plasma concentrations. PK parameters were calculated via commercially available software and measures of exposure were used to assess differences in dosing routes. The table below summarizes exposures as measured by area under the curve (AUC), concentration at its maximum (Cmax), and bioavailability.

Both arms dosed 20 mg/kg in Captex 355

| PK Parameter | PO | ID |
|---|---|---|
| AUC (ng-hr/ml) | 998 | 4943 |
| Cmax (ng/ml) | 243 | 884 |
| F (%) | 21 | 100 |

### Example 6

### In Vitro Assay for Release

The ability of a dosage form to release a compound disclosed herein from a dosage form at a particular pH can be determined by methods well known in the art. For example, effectiveness of the enteric coated formulation to release a compound of the disclosure at the desired pH can be determined by a conducting disintegration and dissolution study in a calibrated USP Apparatus 1 or 2, with and without surfactant set at the appropriate stirring rate and temperature. Solubility is determined initially at lower pH, for example in 0.1 N HCl, for a period of time (for example 2 h) at 37° C. to determine if any drug has been released. The enteric coating is considered acceptable if <10% of drug is released in low pH medium. The pH of the medium is then adjusted to the desired pH (for example to pH 5.5) with a buffer at 37° C. at which point the enteric coating should disintegrate and release the drug in the medium. Samples are removed and analyzed for concentration of the drug at a predetermined time points (for example, 15, 30, 45 minutes, etc.). Solubility (concentration) of a compound of the disclosure can be determined using UV spectroscopy or by HPLC equipped with a UV detector against a predetermined concentration curve using the Reference Standard. To enhance the solubility of the drug, a surfactant (for example Tween 80, TPGS, SLS) can be added to the medium.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

What is claimed:

1. A solid oral dosage form comprising:
(i) ibrutinib and/or a pharmaceutically acceptable salt thereof;
(ii) means for release of said ibrutinib and/or said pharmaceutically acceptable salt thereof in the intestine; and
(iii) at least one pharmaceutically acceptable excipient.

2. A solid oral dosage form comprising:
(i) ibrutinib and/or a pharmaceutically acceptable salt thereof;
(ii) means for increasing oral bioavailability, as measured by the area under the curve (AUC), of said ibrutinib or said pharmaceutically acceptable salt thereof as compared to the oral bioavailability obtained from an immediate release solid oral dosage form comprising the same dose of said ibrutinib and/or said pharmaceutically acceptable salt thereof; and
(iii) at least one pharmaceutically acceptable excipient.

3. A solid oral dosage form comprising:
(i) ibrutinib and/or a pharmaceutically acceptable salt thereof;
(ii) at least one coating chosen from enteric coatings and non-enteric time-delayed release coatings; and
(ii) at least one pharmaceutically acceptable excipient.

4. The solid oral dosage form of claim 3 wherein said dosage form is coated with said at least one coating chosen from enteric coatings and non-enteric time-delayed release coatings.

5. The solid oral dosage form of claim 3 wherein said ibrutinib and/or said pharmaceutically acceptable salt thereof is coated with at least one coating chosen from enteric coatings and non-enteric time-delayed release coatings.

6. The solid oral dosage form of any of claims 3 to 5 wherein the said at least one coating is chosen from enteric coatings.

7. The solid oral dosage form of claim 6 wherein the said enteric coatings are chosen from polymeric coatings.

8. The solid oral dosage form of claim 7 wherein the said polymeric coatings are chosen from polymethacrylates, cellulose-based polymers, and polyvinyl derivatives.

9. The solid oral dosage form of any of claims 1 to 8 wherein said solid oral dosage form contains from about 20 mg to about 450 mg of said ibrutinib and/or said pharmaceutically acceptable salt thereof.

10. The solid oral dosage form of any of claims 1 to 8 wherein said solid oral dosage form contains from about 20 mg to about 420 mg of said ibrutinib and/or said pharmaceutically acceptable salt thereof.

11. The solid oral dosage form of any of claims 1 to 8 wherein said solid oral dosage form contains from about 20 mg to about 300 mg of said ibrutinib and/or said pharmaceutically acceptable salt thereof.

12. The solid oral dosage form of any of claims 1 to 8 wherein said solid oral dosage form contains from about 50 mg to about 220 mg of said ibrutinib and/or said pharmaceutically acceptable salt thereof.

13. The solid oral dosage form of any of claims 1 to 12 wherein said solid oral dosage form is chosen from tablets and capsules.

14. The solid oral dosage form of any of claims 1 to 13 wherein said at least one pharmaceutically acceptable excipient is chosen from binders, surfactants, diluents, buffering agents, antiadherents, glidants, polymers, retardants, disintegrants, antioxidants, antifoaming agents, fillers, flavors, colors, lubricants, sorbents, preservatives, plasticizers, and sweeteners.

15. A solid oral dosage form comprising:
(i) about 20 mg to about 450 mg of ibrutinib and/or a pharmaceutically acceptable salt thereof;
(ii) at least one coating chosen from an enteric coating and a non-enteric time-delayed release coating; and
(iii) at least one pharmaceutically acceptable excipient;
wherein said oral dosage form increases the oral bioavailability, as measured by the area under the curve (AUC), of said ibrutinib or said pharmaceutically acceptable salt thereof by at least 20% as compared to the bioavailability obtained from an immediate release solid oral dosage form comprising the same dose of said ibrutinib and/or said pharmaceutically acceptable salt thereof.

16. The solid oral dosage form of claim 15 wherein said solid oral dosage form increases the oral bioavailability by at least 50%.

17. The solid oral dosage form of claim 15 wherein said solid oral dosage form increases the bioavailability by at least 75%.

18. The solid oral dosage form of claim 15 wherein said solid oral dosage form increases the bioavailability by at least 95%.

19. A method of increasing oral bioavailability of ibrutinib and/or a pharmaceutically acceptable salt thereof in a patient in recognized need thereof, which method comprises administering said ibrutinib and/or said pharmaceutically acceptable salt thereof to said patient a solid oral dosage form of any of claims 1 to 18.

20. A method of treating a disease treatable by inhibition of a tyrosine kinase in a patient in recognized need thereof which method comprises administering to said patient, in single or multiple doses, a therapeutically effective amount of ibrutinib and/or a pharmaceutically acceptable salt thereof in a dosage form that releases said ibrutinib and/or said pharmaceutically acceptable salt thereof in the intestine.

21. The method of claim 20 wherein the tyrosine kinase is chosen from BLK, BMX, EGFR, HER2, HER4, ITK, TEC, BTK, and TXK.

22. The method of claim 20 wherein the tyrosine kinase is BTK.

23. The method of any of claims 20 to 22 wherein said ibrutinib and/or said pharmaceutically acceptable salt thereof is released in the small intestine.

24. The method of any of claims 20 to 23 wherein said therapeutically effective amount of said ibrutinib and/or said pharmaceutically acceptable salt thereof is from about 20 mg/day to about 450 mg/day.

25. The method of claim 24 wherein said therapeutically effective amount of said ibrutinib and/or said pharmaceutically acceptable salt thereof is from about 30 mg/day to about 300 mg/day.

26. The method of claim 24 wherein said therapeutically effective amount of said ibrutinib and/or said pharmaceutically acceptable salt thereof is from about 50 mg/day to about 220 mg/day.

27. The method of any of claims 20 to 26 wherein said solid dosage form comprises at least one coating chosen from enteric coatings and non-enteric time-delayed release coatings; and at least one pharmaceutically acceptable excipient.

28. The method of claim 27 wherein said dosage form is coated with said at least one coating chosen from enteric coatings and non-enteric time-delayed release coatings

29. The method of claim 27 wherein said ibrutinib or said pharmaceutically acceptable salt thereof is coated with at least one coating chosen from enteric coatings and non-enteric time-delayed release coatings.

30. The method of any of claims 27 to 29 wherein the said at least one coating is chosen from an enteric coating.

31. The method of claim 30 wherein the said enteric coatings are polymeric coatings chosen from polymethacrylates, cellulose-based polymers, and polyvinyl derivatives.

32. The method of any of claims 20 to 31 wherein the disease is selected from an autoimmune disease, cancer and an inflammatory disease.

33. The method of any of claims 20 to 31, wherein the disease is leukemia or lymphoma.

34. The method of claim 33 wherein the leukemia is chosen from chronic lymphocytic leukemia (CLL), small lymphocytic lymphoma (SLL), multiple myeloma, mantle cell lymphoma, and B-cell non-Hodgkin lymphoma.

35. The method of any of claims 20 to 34 wherein said dosage form is administered in combination with at least one additional agent chosen from anti-inflammatory and antiproliferative agents.

36. The method of any of claims 20 to 34 wherein said dosage form is administered in combination with at least one additional agent chosen from of atumumab, bendamustine, and rituaximab.

37. The method of claim 35 or 36 wherein the combination administration is simultaneously or sequentially.

38. The method of any of claims 20 to 35 wherein said ibrutinib and/or said pharmaceutically acceptable salt thereof is released from said dosage form at or above about pH 5.
